(19) United States
(12) Patent Application Publication
(10) Pub. No.: US 2018/0149402 A1
(43) Pub. Date: May 31, 2018

(54) COOLING ARTICLES AND COOLING SYSTEMS

(71) Applicant: Hill-Rom Services, Inc., Batesville, IN (US)

(72) Inventors: Varad Narayan Srivastava, Batesville, IN (US); David Lawrence Bedel, Batesville, IN (US); David Lance Ribble, Batesville, IN (US); David Newkirk, Batesville, IN (US); Roger Bonenfant, Skaneateles Falls, NY (US); Craig Meyerson, Skaneateles Falls, NY (US); Jeffrey C. Marrion, Batesville, IN (US)

(73) Assignee: Hill-Rom Services, Inc., Batesville, IN (US)

(21) Appl. No.: 15/801,877

(22) Filed: Nov. 2, 2017

Related U.S. Application Data
(60) Provisional application No. 62/426,761, filed on Nov. 28, 2016.

(51) Int. Cl. F25D 5/02; F25B 21/02; A61F 7/10

(52) U.S. Cl. CPC F25D 5/02; A61F 2007/0276; A61F 7/106; F25B 21/02

(57) ABSTRACT

Embodiments include cooling articles including a receptacle, an endothermic ingredient contained in a first capsule within the receptacle, an endothermic matrix within the receptacle, and a color indicator. The endothermic ingredient, when mixed with the endothermic matrix, generates an endothermic reaction, and the color indicator is activated upon the generation of the endothermic reaction to indicate that cooling has been initiated. Other embodiments include cooling articles including an electric circuit configured to melt at least a portion of the first capsule to enable the endothermic ingredient and the endothermic matrix to mix. Systems for providing cooling that include a person support apparatus and a cooling article including a thermoelectric device coupled to the person support apparatus to direct heat from a source region to a sink region including the person support apparatus are also described.

### CROSS-REFERENCE TO RELATED APPLICATIONS

The present application claims priority to U.S. Provisional Patent Application Ser. No. 62/426,761, filed Nov. 28, 2016, and entitled “Cooling Articles and Cooling Systems,” the entirety of which is incorporated by reference herein.

### TECHNICAL FIELD

The present specification generally relates to cooling articles and cooling systems, and more specifically, to cooling articles and cooling systems having cooling features such as flexible thermoelectric elements and/or chemical agents contained within separate capsules that may be broken to enable the generation of an endothermic reaction.

### BACKGROUND

Conventionally, an individual may be positioned on a person support surface during and after a surgical procedure. Areas of the individual in contact with the person support surface can increase in temperature after extended periods of time, such as during recovery and/or rehabilitation. The increase in temperature may result in moisture (e.g., perspiration) becoming trapped between the person support surface and the individual's skin. The combination of increased temperature and moisture for extended periods of time can lead to the development of pressure ulcers. Moreover, it is known that cooling may reduce swelling an inflammation surrounding a wound and assist in healing.

Accordingly, a need exists for alternative cooling articles and cooling systems.

### SUMMARY

According to some embodiments of the present disclosure, an article for cooling includes a receptacle, an endothermic ingredient contained in a first capsule within the receptacle, an endothermic matrix within the receptacle, and at least one encapsulated dye. When the endothermic ingredient is mixed with the endothermic matrix, an endothermic reaction is generated. The at least one encapsulated dye is released upon the generation of the endothermic reaction to indicate that cooling has been initiated.

According to some embodiments of the present disclosure, an article for cooling includes a fluid impermeable receptacle, an endothermic ingredient contained in a first capsule within the fluid impermeable receptacle, an endothermic matrix within the fluid impermeable receptacle, and an electric circuit configured to melt at least a portion of the first capsule to enable the endothermic ingredient and the endothermic matrix to mix. When mixed with the endothermic matrix, the endothermic ingredient generates an endothermic reaction.

According to some embodiments, a system for providing cooling includes a person support apparatus and a cooling apparatus. The cooling apparatus includes a thermoelectric device and is coupled to the person support apparatus. Accordingly, the cooling apparatus directs heat from a source region proximate a support surface of the cooling apparatus to a sink region that includes the person support apparatus.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

Referring now to the illustrative examples in the drawings, wherein like numerals represent the same or similar elements throughout:

FIG. 1 schematically depicts an example cooling article according to one or more embodiments shown and described herein;

FIG. 2A schematically depicts an example cross-section of a cooling article according to one or more embodiments shown and described herein;

FIG. 2B schematically depicts another example cross-section of a cooling article according to one or more embodiments shown and described herein;

FIG. 2C schematically depicts another example cross-section of a cooling article according to one or more embodiments shown and described herein;

FIG. 3 schematically depicts a cross-section of a cooling article including a flexible thermoelectric module according to one or more embodiments shown and described herein;

FIG. 4A schematically depicts another example cooling article according to one or more embodiments shown and described herein;

FIG. 4B schematically depicts a cross-section of the cooling article depicted in FIG. 4A along line B-B according to one or more embodiments shown and described herein;

FIG. 4C schematically depicts an example electronic circuit that can be employed in the cooling article depicted in FIG. 4A according to one or more embodiments shown and described herein;

FIG. 5A schematically depicts another example cooling article according to one or more embodiments shown and described herein;

FIG. 5B schematically depicts an RFID system that may be incorporated into the cooling article depicted in FIG. 5A according to one or more embodiments shown and described herein;

FIG. 6 schematically depicts a person support apparatus having a cooling article disposed thereon according to one or more embodiments shown and described herein;

FIG. 7 schematically depicts a cross-section of a person support apparatus having a cooling article disposed thereon according to one or more embodiments shown and described herein; and

FIG. 8 schematically depicts a person support apparatus including a vibration therapy surface according to one or more embodiments shown and described herein.

### DETAILED DESCRIPTION

FIG. 1 generally depicts one embodiment of a cooling article. The cooling article may provide localized cooling to an individual in contact with the cooling article, such as to aid in the prevention of pressure ulcers. In various embodiments described herein, the cooling article includes at least one endothermic ingredient that, when mixed with an endothermic matrix, generates an endothermic reaction which provides a cooling effect. Various embodiments of the cooling article, systems including the cooling article, and methods for activating the cooling article will be described in more detail herein.

### Cooling Articles

In FIG. 1, a cooling article 100 is depicted. The cooling article 100 may be in the form of a cooling mat, a cooling pad, or the like. For example, the cooling article 100 may be in the form of a cooling mat to be positioned between an individual and a person support apparatus, or may be in the form of a wearable pad. The cooling article 100 may have a variety of shapes, including but not limited to, a shape designed for application of cooling to the sacral region, a heel, an elbow, or the like. Regardless of the particular form, the cooling article 100 generally includes a first surface 102 and a second surface 104.

According to various embodiments, the first surface 102 may be made of an absorbent fibrous sheet or absorbent web, such as those employed in facial tissue, bath tissue, paper towels, or the like. In such embodiments, the first surface 102 may absorb moisture (e.g., perspiration) and other bodily fluids, and remove them from the surface of the cooling article 100, as will be described in greater detail hereinbelow. In other embodiments, the first surface 102 may be made of a substantially fluid impermeable material such as, by way of example and not limitation, a coated woven or non-woven nylon or polyester. The nylon or polyester may be coated on one or both sides with a thermoplastic polyurethane or polyurethane to make the material fluid impermeable. In still other embodiments, the first surface 102 may be made of a thermally conductive elastomer, such as those commercially available under the trade name CoolPoly® from Cool Polymers, Inc. (Warwick, R.I.), which may form a barrier surface and spread the cooling generated by the cooling article 100, thereby increasing the efficiency of the cooling article 100. Still other materials may be employed in other embodiments, provided that the materials enable the cooling effect to be observed on external to the cooling article 100.

The second surface 104 may be made of the same material as the first surface 102, or the second surface 104 may be made of a different material. In some embodiments, the second surface 104 includes an adhesive to enable the cooling article 100 to be secured in place. For example, the second surface 104 may include an adhesive to enable the cooling article 100 to be removably secured to a person support apparatus or to itself. In other embodiments, the adhesive may be included on the first surface 102 of the cooling article 100, such that the cooling article 100 may be adhered to an individual wearing the cooling article 100.

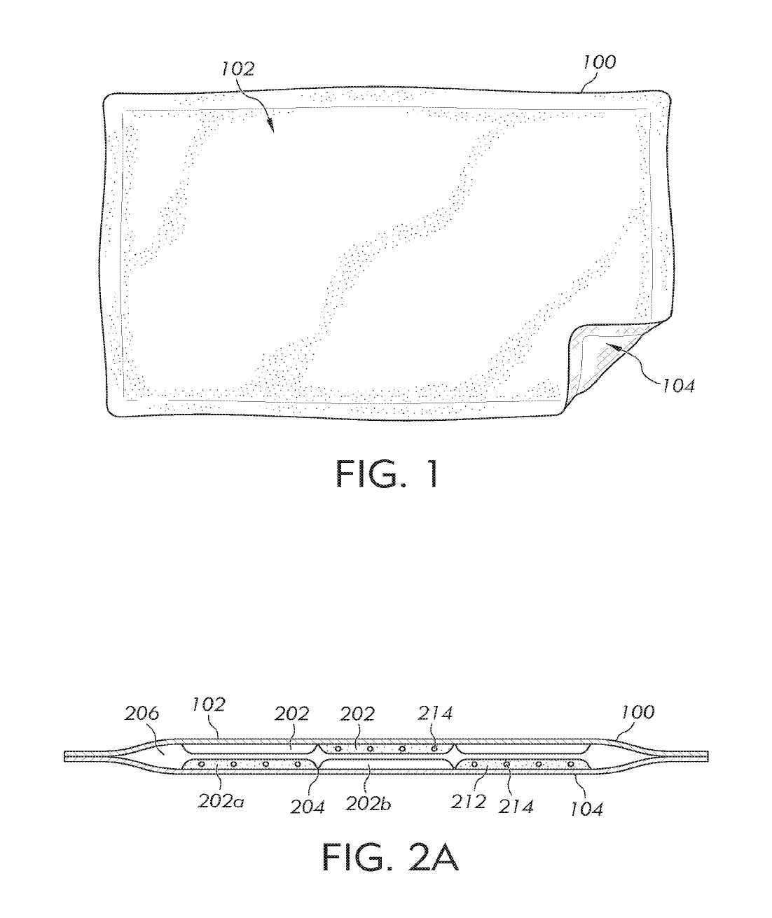

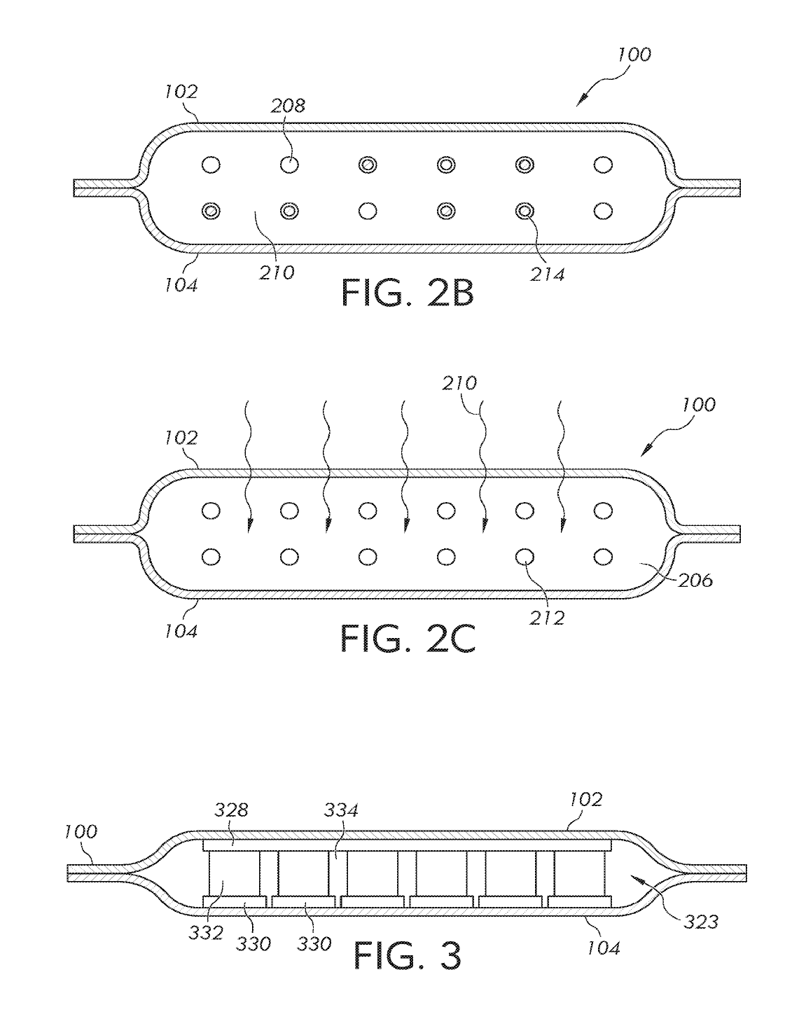

In various embodiments, the first and second surfaces 102, 104 may be joined around at least a periphery of the cooling article 100 to form a receptacle 206 (see FIG. 2A) for containing one or more cooling features. In other embodiments, the first and second surfaces 102, 104 may be joined around the periphery of the cooling article 100 and joined at various locations along the length and width of the cooling article 100, such as to form a cooling article 100 having multiple chambers or receptacles.

According to various embodiments, one or more cooling features are provided within the receptacle(s) formed by the first and second surfaces 102, 104. In various embodiments, such as the embodiments depicted in FIGS. 2A-2C, the cooling feature includes one or more chemical agents to generate an endothermic reaction. In other embodiments, such as the embodiment depicted in FIG. 3, a flexible thermoelectric module may be incorporated in the receptacle. In still other embodiments, the cooling article may include both a flexible thermoelectric module and one or more chemical agents. Each of these types of cooling features will be discussed in turn.

In embodiments in which the cooling feature includes one or more chemical agents, the chemical agents may include one or more endothermic ingredients and an endothermic matrix. A variety of endothermic ingredients may be used, provided it slowly absorbs heat when dissolved in an endothermic matrix. The endothermic ingredient may include, by way of example and not limitation, inorganic salt or inorganic hydrates of ammonia, alkali metals, calcium, urea, simple saccharides and mixtures thereof. Non-limiting examples of suitable inorganic salt include crystalline phosphates, sulfates, carbonates, nitrates, and the like. Sodium phosphate salts, sodium ammonium phosphate salts, and ammonium phosphate salts may be employed in some embodiments. Examples of sodium phosphate salts are disodium hydrogen phosphate, sodium dihydrogen phosphate, trisodium phosphate, and hydrates thereof. Examples of sodium ammonium phosphate salts and ammonium phosphate salts are sodium ammonium hydrogen phosphate, diammonium hydrogen phosphate, ammonium dihydrogen phosphate, triammonium phosphate, and hydrates thereof. These salts may be used singly or in combinations of two or more. In other embodiments, potassium chloride, ammonium nitrate, sodium nitrate, ammonium chloride, calcium chloride, or lithium chloride may be employed. Additional examples of inorganic salts that may be suitable include, by way of example and not limitation, sodium carbonate, sodium hydrogen carbonate, potassium sodium carbonate, sodium chromium carbonate, sodium scandium carbonate, sodium cerium carbonate, sodium sulfate, and hydrates thereof. In some embodiments, the endothermic ingredient includes at least ammonium nitrate.

In various embodiments, the endothermic matrix is water. However, endothermic reactions are not limited to the dissolution of endothermic ingredients in water. Endothermic reactions occur when a reaction requires more energy to break the bonds of the reactants than energy given off in forming new bonds. As the reaction progresses, energy is absorbed from the surroundings and a decrease in temperature is observed. Thus, solid/solid chemical reactions can occur which are endothermic.

Examples of ingredients used for such reaction would involve hydrated inorganic salts in their solid form reacting with selected solid ammonium salts. For example, the mixture of barium hydroxide octahydrate with ammonium chloride generates an endothermic reaction.

In some embodiments, solid/solid endothermic reactions involving hydrates may combined with the presence of one or more endothermic ingredients mentioned above. As the two solid portions combine bonds of hydrated water are broken and water is released, an endothermic reaction is initiated. Then, the presence of liquid water provides a solvent for a second or third endothermic ingredient to dissolve, which may increase the duration of the cooling and/or further lower the temperature.

In various embodiments, at least the endothermic ingredient is contained within a capsule or chamber within the receptacle. For example, the endothermic ingredient may be contained within a capsule within the receptacle while the endothermic matrix is provided within the receptacle, or the endothermic ingredient and the endothermic matrix may be contained within capsules within the receptacle. In embodiments in which the endothermic ingredient and the endothermic matrix are both contained within capsules, the endothermic ingredient and the endothermic matrix are contained within separate capsules. For example, in the embodiment depicted in FIG. 2A, a cooling article 100 includes a plurality of capsules 202. The plurality of capsules 202 includes individual capsules 202a, 202b, etc. The individual capsules of the plurality of capsules are separated from one another by barriers. For example, capsule 202a is separated from an adjacent capsule 202b by a barrier 204. The capsule 202a may include the endothermic ingredient while the adjacent capsule 202b includes the endothermic matrix.

Accordingly, to activate cooling of the cooling article 100, the capsules within the receptacle 206 are broken to enable the endothermic ingredient contained in capsule 202a and the endothermic matrix contained in capsule 202b to contact one another, as will be described in greater detail hereinbelow. In some embodiments, the entire capsule may be broken, enabling the endothermic ingredient and the endothermic matrix to be mixed within the receptacle 206. However, in other embodiments, the barrier 204 between the capsules is broken. In such embodiments, the endothermic reaction may be contained within the capsules instead of occurring within the receptacle 206. In other embodiments, a portion of the capsule offset from the barrier 204 is broken.

In various embodiments, the capsules containing at least the endothermic ingredient are microcapsules. As used herein, the term “microcapsules” includes microspheres, beads, and the like. The microcapsules may be formed from thermoplastics, paraffin or another type of wax, or another suitable type of encapsulator. As depicted in FIG. 2B, the cooling article 100 may include microcapsules 208 containing the endothermic ingredient that are dispersed within the endothermic matrix 210. As above, to activate cooling, the microcapsules 208 may be burst to release the endothermic ingredient into the endothermic matrix 210. In other embodiments, both the endothermic ingredient and the endothermic matrix may be contained in microcapsules that, when burst, enable the endothermic ingredient and endothermic matrix to react together.

In still other embodiments, such as the embodiment depicted in FIG. 2C, the endothermic ingredient 212 may be contained within the receptacle 206 and activated when the endothermic matrix 210 is absorbed by the cooling article 100. For example, the endothermic matrix 210 may be moisture or bodily fluid, such as urine or perspiration, that is absorbed by the first surface 102 of the cooling article 100. In such embodiments, the first surface 102 is permeable to fluid to enable the endothermic matrix 210 to enter the receptacle 206, but may be impermeable to the endothermic ingredient 212 such that the endothermic ingredient 212 does not contact an individual in contact with the first surface 102. For example, the first surface 102 may be made of a thin film that allows heat transfer and contains the chemical agent(s), but is moisture permeable. As another example, the first surface 102 may be made of a cellulose phosphate paper to prevent the chemical agents from soaking through the first surface 102. Accordingly, the first surface 102 may provide absorption while protecting against chemical agents contained within the cooling article 100 from contacting an individual in contact with exterior of the cooling article 100.

In various embodiments, the cooling article 100 may further include an indicator to indicate when cooling has been activated. The indicator may be, in various embodiments, a color indicator formed from a dye or other chemical. In embodiments, the color indicator includes at least one encapsulated dye 214 that may be released upon the generation of the endothermic reaction to indicate that cooling has been initiated. The encapsulated dye 214 may be, for example, encapsulated within the capsule 202a with the endothermic ingredient 212. Accordingly, when the capsule 202a is burst, the dye 214 is released along with the endothermic ingredient 212. Alternatively, the encapsulated dye 214 may be encapsulated within its own capsule(s). For example, the encapsulated dye 214 may be contained within microcapsules, such as the microcapsules depicted in FIG. 2B, which may be burst along with microcapsules containing the endothermic ingredient. As with the capsules containing the endothermic ingredient and/or the endothermic matrix described hereinabove, the capsules containing the dye 214 may be burst responsive to the application of pressure, the application of heat, or other methods. For example, the dye 214 may be encapsulated using paraffin or another wax, which may be melted to release the dye 214.

In still other embodiments, the dye may be activated by the endothermic matrix to indicate that cooling has been initiated. For example, the dye may be colorless and produce a color responsive to a chemical reaction that takes place when the dye is exposed to the endothermic matrix. More particularly, the dye may be a colorless color-developing agent formed of an electron-donating coloration compound that reacts with water to develop a color. The color-developing agent may contain at least one of rhodamine B lactam, 6-diethylamino-benzo[a]fluoran, 3-diethylamino-benzo[a]fluoran, 3-diethylamino-7,8-benzo[a]fluoran, 9-diethylamino-benzo[a]fluoran, 3-diethylamino-7-chlorofluoran, 3,3-bis(1-n-butyl-2-methyl-indoyl-3)phthalate, 3,3-bis(1-ethyl-2-methyl-indoyl-3)phthalate, 3,6-bis(diethylamino)fluoran-γ-(4′-nitro) anilinolactam, 3-diethylamino-6-methyl-7-chlorofluoran, 2-bromine-3-methyl-6-dibutylaminofluoran, 1,3-dimethyl-6-diethylaminofluoran, 1,3,3-trimethyl-indolino-7′-chloro-β-naphthospiropyran, 3-cyclohexylamino-6-chlorofluoran, 2-(phenyliminoethanezyliden)-3,3-trimethylindoline, N-acetylauramine, N-phenylauramine, 2-{2-[4-(dodecyloxy)-3-methoxyphenyl]-ethenyl}quinoline, marachite green lactone, 3-diethylamino-7-dibenzoylaminofluoran, 3-diethylamino-7-chloroanilinofluoran, 3,6,5′-tri(diethylamino)fluorene-9-spiro-1′-(3′-isobenzofuran), 2,N,N-dibenzylamino-6-diethylaminofluoran, 3-(N,N-diethylamino)-7-(N,N-dibenzylamino)fluoran, 3-[2,2-bis(1-ethyl-2-methylindoyl-3)vinyl]-3-(4-diethylaminophenyl)-phthalide, 3,3-bis(4-diethylamino-2-ethoxyphenyl)-4-azaphthalide, crystal violet lactone, ethyl leuco methylene blue, methoxybenzoyl leuco methylene blue, di-β-naphthospiropyran, 3,3-bis(4-diethylaminophenyl)-6-diethylaminophthalide, 3-(4-diethylamino-2-ethoxyphenyl)-3-(1-ethyl-2-methylindoyl-3)-4-azaphthalide, 3-(4-diethylaminophenyl)-3-(1-ethyl-2-methylindoyl-3)-phthalide, 3-cyclohexylmethylamino-6-methyl-7-anilinofluoran, 3-diethylamino-6-methyl-7-anilinofluoran, 3-n-dibutylamino-6-methyl-7-anilinofluoran, 3-diethylamino-6-methyl-7-kylindenofluoran, 2-(2-chloroanilino)-6-diethylaminofluoran, 2-(2-chloroanilino)-6-dibutylaminofluoran, 2-anilino-3-methyl-6-diethyllaminofluoran, 2-anilino-3-methyl-6-dibutylaminofluoran, 6-diethylamino-3-methyl-2-(3-toluideno)-fluoran, 6-diethylamino-3-methyl-2-(2,4-kylindeno)-fluoran, 6-diethylamino-3-methyl-2-(2,6-kylindeno)-fluoran.

The dye may alternatively be a hydratable salt that, in its anhydrous condition is a white or lightly-colored powder, but upon contact with water, transforms into a contrasting-colored hydrated compound. For example, copper sulfate may be employed. In still other embodiments, other types of suitable dyes may be employed.

In embodiments including a color indicator, the first surface 102 should be selected from a material that enables the color indicator to be readily observable. For example, the first surface 102 may be made of a transparent or semi-transparent material, a white or light-colored material, or even a material such as cellulose, through which the dye may seep through or stain to enable the color indicator to be observed.

While various embodiments have been described including chemical agents capable of generating an endothermic reaction within a cooling article 100, it is contemplated that the chemical agents may be combined within, contained within, or provided to the cooling article 100 by other methods. For example, one or more of the chemical agents for use in generating the endothermic reaction may be provided to the cooling article 100 just before its activation, the chemical agents may be mixed remotely before being provided to the cooling article via tubes, or the like.

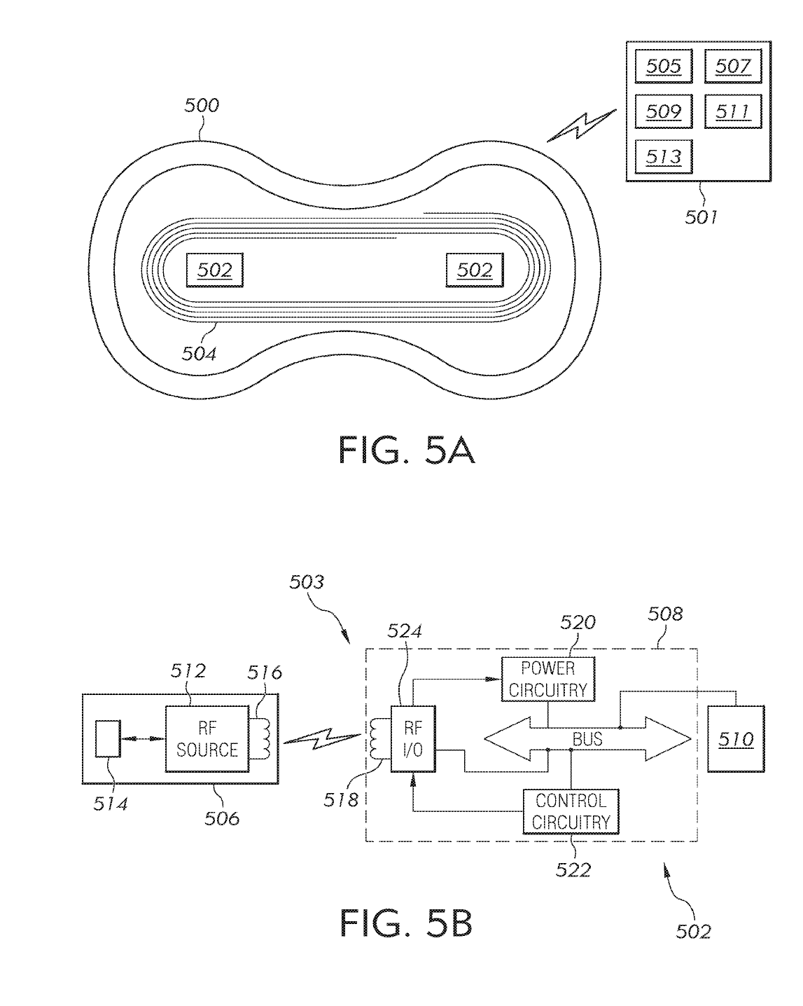

As described hereinabove, in some embodiments, the cooling article 100 may include a flexible thermoelectric module to provide cooling. FIG. 3 depicts a cross-section of a cooling article 100 including a flexible thermoelectric module 323. The flexible thermoelectric module 323 is positioned between the first surface 102 and a second surface 104 of the cooling article 100. The first surface 102 may form a person support surface, for example, when the cooling article 100 is positioned against a portion of an individual. The flexible thermoelectric module 323 includes an upper substrate 328 and a lower substrate 330. As depicted in FIG. 3, the lower substrate 330 is provided in a plurality of sections to enable the flexible thermoelectric module 323 to flex. The upper substrate 328 and the lower substrate 330 sandwich one or more thermoelectric elements 332 and have electrical conductors that connect the thermoelectric elements 332. For example, the thermoelectric elements 332 may be connected via printed circuits (not shown) or via the RF coil 504 depicted in FIG. 5A. In embodiments in which the RF coil 504 is used to connect the thermoelectric elements 332, the thermoelectric elements 332 may be powered by RF energy, as described in accordance with FIG. 5B. Alternatively, the thermoelectric elements 332 may be connected to one or more lead wires and/or lead pads (not shown) that are electrically coupled to an external power source. In various embodiments, the upper substrate 328 and/or the lower substrate 330 may be made of a rigid material, although in other embodiments, one or more of the substrates may be made of a flexible material.

In embodiments, the flexible thermoelectric module 323 is configured to draw heat away from the first surface 102 (e.g., a source region), such as heat produced by an individual in contact with the first surface 102, and toward the second surface 104 (e.g., a sink region). The second surface 104, as will be described in greater detail hereinbelow, may be in contact with a heat sink to further remove the heat from the individual.

In various embodiments, the flexible thermoelectric module 323 may further be configured to provide heat to an individual in contact with the first surface 102. For example, the current running through the thermoelectric elements 332 may be reversed to draw heat from the second surface 104 and provide it to the first surface 102. Accordingly, the cooling article 100 may provide both heating and cooling to the individual.

Although embodiments have been described in which the cooling feature of the cooling article 100 includes either chemical agents (FIGS. 2A-2C) or thermoelectric modules (FIG. 3), it is further contemplated embodiments may include both chemical agents and thermoelectric modules. For example, chemical agents may be employed to generate an endothermic reaction to act as a heat sink to remove heat from the thermoelectric module.

### Methods of Activating Cooling Articles

As described hereinabove, various embodiments provide articles including cooling features that may be activated to provide cooling, such as to an individual in contact with the article. These cooling articles may be activated in a variety of ways, various embodiments of which will now be described.

Returning to FIG. 2A, an endothermic ingredient is provided in a capsule 202a, which is separated from an adjacent capsule 202b which contains an endothermic matrix by a barrier 204, as described hereinabove. In order to activate the cooling article 100, the capsules 202 are broken, enabling the endothermic ingredient to mix with the endothermic matrix in the receptacle 206 between the first surface 102 and the second surface 104 of the cooling article 100.

According to various embodiments, the capsules 202, and/or the barrier 204 between a capsule 202a and an adjacent capsule 202b, may be broken by the application of pressure to one or more of the capsules 202. For example, an individual may apply a pressing force to the cooling article 100 sufficient to break the capsules 202 and/or the barrier 204, such as with the individual's hands or body. In some embodiments, the applied pressure may be the result of an individual sitting or lying on the cooling article 100, such as when the cooling article 100 is positioned between the individual and a person support apparatus. In various embodiments, the cooling article 100 may have one or more predefined break points along which the capsules 202 may break. For example, a portion of the capsule 202 may be thinner or otherwise weakened to enable the capsule 202 to be broken upon application of pressure from the individual.

In various embodiments, some capsules 202 within the cooling article 100 may break at lower pressures than other capsules. In such embodiments, the amount of pressure applied by the individual may be used to control the amount of cooling provided by the cooling article 100. For example, an initial application of pressure from the individual may break only about 10% or 25% of the capsules within the cooling article 100 while a subsequent application of pressure from the individual may break the remaining capsules to release additional amounts of the endothermic ingredient and endothermic matrix, thus providing additional cooling.

In embodiments in which the application of pressure from an individual is utilized to break the capsules 202, the capsules 202 are formed such that they may be broken upon application of from about 20 Newtons (N) to about 50 N of force.

In other embodiments, such as the embodiment depicted in FIG. 2B, the endothermic ingredient is contained within microcapsules 208 dispersed within the endothermic matrix 210. In such embodiments, cooling is initiated when the microcapsules 208 are burst to release the endothermic ingredient into the endothermic matrix 210. In some embodiments, the microcapsules 208 may be burst upon application of a preset load. The preset load may be, for example, the application of pressure from an individual, as described above.

In other embodiments, cooling may be initiated by means other than application of pressure. In embodiments in which the microcapsules 208 are formed from paraffin or other wax, the microcapsules 208 may be burst in response to exposure to a particular temperature. For example, the microcapsules 208 may be formed from a material selected to melt at a particular temperature. The temperature may be, for example, 37° C. (e.g., body temperature) or greater. As the microcapsules 208 melt, the endothermic ingredient is released into the endothermic matrix 210. Accordingly, when an individual is in contact with the cooling article 100, his or her body temperature may cause the microcapsules 208 to melt, releasing the endothermic ingredient into the endothermic matrix 210 and initiating cooling.

In still other embodiments, such as the embodiment depicted in FIG. 2C, cooling may be activated by exposure to moisture. In FIG. 2C, an endothermic ingredient 212 is contained within the receptacle 206 of the cooling article 100. As described hereinabove, the endothermic matrix 210 may be absorbed through the first surface 102 of the cooling article 100 to activate cooling. In one particular embodiment, the cooling article 100 may be in the form of an incontinence pad. In response to an incontinence event, the moisture (e.g., urine) is absorbed by the first surface of the incontinence pad and reaches the endothermic ingredient, which may be, for example, ammonium nitrate, initiating an endothermic reaction. The endothermic reaction provides cooling to the skin of the individual in contact with the incontinence pad until the individual is cleaned and the incontinence is removed. The cooling of the skin may, for example, provide protection of the skin from damage that may result from prolonged exposure to moisture and the development of pressure ulcers.

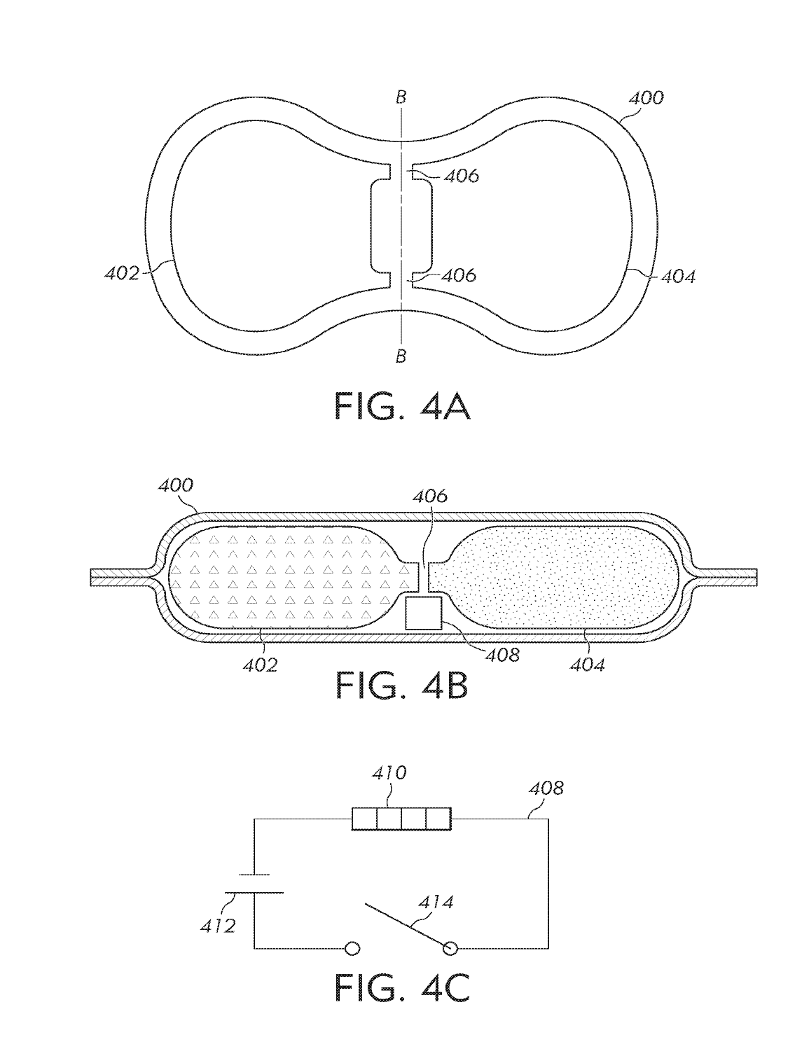

Various embodiments may employ electricity to melt a barrier between capsules containing the endothermic ingredient and the endothermic matrix. FIG. 4A depicts a cooling article in the form of a sacral cooling pad 400 that includes a first capsule 402 and a second capsule 404 separated from one another by a low temperature barrier 406. The first capsule 402 may include, for example, the endothermic ingredient while the second capsule may include, for example, the endothermic matrix. FIG. 4B shows a cross-section of the sacral cooling pad 400 of FIG. 4A along line B-B. FIG. 4B further depicts an electrical circuit 408 that is configured to provide an electrical charge sufficient to heat the low temperature barrier 406 to or past its melting point. When the low temperature barrier 406 melts, the endothermic ingredient contained in the first capsule 402 and the endothermic matrix contained in the second capsule 404 can mix with one another to generate an endothermic reaction.

FIG. 4C depicts the electrical circuit 408 in greater detail. As shown in FIG. 4C, the electrical circuit 408 includes at least a heating element 410, a power source 412, and a switch 414. The heating element 410 may be a resistor, a heating coil, or another element configured to convert electricity into heat. In various embodiments, the heating element 410, as part of the electrical circuit 408, enables small areas of the low temperature barrier 406 to be heated for a brief period of time sufficient to melt the low temperature barrier 406. The power source 412 may be an external power source connected to the sacral cooling pad 400, such as through lead pads (not shown), an RFID system, such as described below in accordance with FIG. 5B, or a battery, such as a thin film battery.

The switch 414 in the electrical circuit 408 may be activated by a variety of methods. For example, a user interface, such as a remote control or other electronic device, may receive a user input to activate cooling, may be removably coupled to the electrical circuit 408 to send a signal to close the switch 414, completing the electrical circuit 408, and causing the heating element 410 to provide heat sufficient to melt the low temperature barrier 406, thus activating the cooling. Alternatively, a Bluetooth-enabled chip (not shown), a chip and battery, or another type of on-board pre-programmed chip may be used to provide a signal to close the switch 414 and activate cooling. In still other embodiments, such as cooling articles including a plurality of capsules and electrical circuits, the electrical circuits may be time-released. For example, electrical circuits may be programmed to be activated at various time intervals to provide cooling over an extended period of time. In other embodiments, the electrical circuit 408 may be an RFID circuit and RFID may be used to trigger the electrical circuit 408 to provide heat to melt the low temperature barrier 406. In even other embodiments, the electrical circuit 408 may be activated by a computer, such as in response to a reading from a temperature or pressure sensor. The use of sensors is described in greater detail below.

In embodiments in which an electrical circuit 408 is employed to melt the low temperature barrier 406, the electrical circuit 408 may be laminated between protective sheets to protect the electrical circuit 408 from exposure to the chemical agents used to generate the endothermic reaction. In other embodiments, various portions of the electrical circuit 408 may be located remotely from the chemical agents for protection.

Although depicted in FIG. 4B as being located within the sacral cooling pad 400, it is contemplated that in some embodiments, the electrical circuit 408 may be located external to the sacral cooling pad 400 or otherwise separable from the sacral cooling pad 400. Accordingly, the sacral cooling pad 400 may be disposed while the electrical circuit 408 may be retained and used with another sacral cooling pad, for example.

Turning now to FIG. 5A, another embodiment of a cooling article in the form of a sacral cooling pad 500 is shown. In FIG. 5A, the sacral cooling pad 500 includes multiple sensors, depicted as RFID sensors 502, and an integrated RF coil 504 for RFID power. Although the sensors in FIG. 5A are RFID sensors, it is contemplated that non-RFID sensors may be incorporated into any of the embodiments described above or below. Sensors may be used, for example, to sense temperature, pressure, moisture, or the like. As will be described, the sensors may provide feedback, such as feedback to activate cooling, or monitoring functionality.

In general, the sensor may be electrically coupled to a computing device 501. Accordingly, the sensor may generate an output which is transmitted to the computing device 501 which may then use the output from the sensor in one or more processes. For example, the computing device 501 may use the output to determine whether cooling should be activated, a level of cooling to be activated, provide a graphical display containing information from the sensor, or the like. The computing device 501 may be any type of computing device, which generally includes at least a processor 505 and a non-transitory memory component 507 for storing one or more computer readable instructions that are executable by the processor 505. The computing device 501 may be coupled to the sensor(s) via wires or wirelessly, as shown in FIG. 5A through networking hardware 509. The computing device 501 may further include a display device 511 and one or more user input devices 513 to enable a user, such as a caregiver, to interact with the sensor through the computing device 501.

In embodiments including a temperature sensor, the temperature sensor may be thermally isolated from the cooling feature of the cooling article, such as through the use of an insulating material known to those skilled in the art. Isolation of the temperature sensor from the cooling feature may enable the temperature sensor to more accurately sense the temperature of, for example, the skin of an individual in contact with the cooling article, rather than sensing the temperature of the cooling feature. The temperature sensor may provide information used, for example, by a computing device, such as the computing device 501, to determine whether cooling should be initiated and/or what level of cooling should be employed. For example, some embodiments may include multiple cooling features or multiple capsules that can be broken at different times to provide varying levels of cooling. The temperature sensor may provide information to enable the computing device 501 to determine how many cooling features to activate and/or how many capsules to break. The temperature sensor may also provide information used to monitor the individual. For example, the temperature sensor may provide an output that is displayed on a graphical user interface (GUI) via the display device 511 to indicate to a caregiver the temperature of the individual in contact with the cooling article.

In embodiments including a pressure sensor, the pressure sensor may enable cooling to be initiated in response to pressure. For example, the pressure sensor may detect the pressure applied to the cooling article and, responsive to determining that the detected pressure exceeds a threshold pressure, may activate cooling. In particular, the pressure sensor may provide information used, for example, by a computing device, such as the computing device 501, to determine whether cooling should be initiated and/or what level of cooling should be employed. The pressure sensor may provide information to enable the computing device 501 to determine how many cooling features to activate and/or how many capsules to break. The pressure sensor may also provide information used to monitor the individual. For example, the pressure sensor may provide an output that is displayed on a graphical user interface (GUI) via the display device 511 to indicate to a caregiver the temperature of the individual in contact with the cooling article.

In embodiments including a moisture sensor, the moisture sensor may enable cooling to be initiated in response to moisture. For example, the moisture sensor may detect the presence of moisture, such as perspiration or an incontinence event, and, responsive to sensing moisture, may activate cooling. In particular, like the pressure sensor and temperature sensor, the moisture sensor may provide information used, for example, by the computing device 501, to determine whether cooling should be initiated and/or what level of cooling should be employed based on a detected moisture level. The moisture sensor may provide information to enable the computing device 501 to determine how many cooling features to activate and/or how many capsules to break. The moisture sensor may also provide information used to monitor the individual. For example, the moisture sensor may provide an output that is displayed on a graphical user interface (GUI) via the display device 511 to indicate to a caregiver the temperature of the individual in contact with the cooling article.

Other embodiments may include one or more types of sensors. Accordingly, cooling may be initiated based on a combination of input from the sensors. For example, cooling may be initiated responsive to a particular temperature received from the temperature sensor, or from a combination of a temperature received from the temperature sensor and a moisture level received from a moisture sensor. In some embodiments, the computing device coupled to the sensors may employ an algorithm to use the inputs from the sensors to determine whether to activate cooling.

In some embodiments, in addition to providing information to a computing device to enable activation and adjustment of the cooling article 100, the sensors may be used to collect data. In such embodiments, the sensors may provide data to the computing device, which stores the data in its memory. In some embodiments, the computing device may transmit the data to one or more remote computing devices to store the data. Data may be aggregated and, in various embodiments, associated with one or more parameters, such as medical condition, age of the individual, weight of the individual, gender of the individual, and the like. Accordingly, the sensors may provide information that enables a data aggregator to better understand pressure ulcers and other conditions and provide improved solutions based on that understanding.

The sensors of various embodiments may be powered by a variety of power sources. For example, the sensors may be coupled to an external power source via lead pads, or may be powered as part of an RFID system, as in the case of RFID sensors 502.

The block diagram of FIG. 5B illustrates an RFID system 503 including an RFID sensor 502 in accordance with various embodiments. The RFID system 503 includes an RFID interrogator 506 and RFID sensor 502 including RFID circuitry 508 and a sensor 510. The RFID interrogator 506 includes a radio frequency (RF) source 512 and a reader 514. The RF source 512 intermittently or continuously transmits RF energy to the RFID sensor 502. The RF energy transmitted by the RF source 512 may be used to power the RFID sensor 502.

The RFID interrogator 506 includes an inductor 516 that serves as an antenna to transmit RF energy to the RFID sensor 502 and to receive RF signals from the RFID sensor 502. The RFID circuitry 508 includes an inductor 518 used to receive the RF energy from the RFID interrogator 506 and to transmit RF signals from the RFID sensor 502 to the RFID interrogator 506.

In embodiments in which the RFID sensor 502 is powered by the RF energy delivered by the RFID interrogator 506, the RFID circuitry 508 includes power circuitry 520 that converts the RF energy received from the RFID interrogator 506 into power useable by the components of the RFID sensor 502, including the control circuitry 522 and the sensor 510. For example, the power circuitry 520 may convert the RF energy into DC or AC power. In some embodiments, the power circuitry 520 may include a battery or other power source that may be used to power the RFID sensor 502 independent from the RFID interrogator 506.

The control circuitry 522 may be configured to output data from the sensor 510 to the RFID interrogator 506. For example, the data may be transmitted from the RFID sensor 502 to the RFID interrogator 506 as a data stream via the RF input/output circuitry 524 and the inductor 518. The data may be received by the RFID interrogator 506 and interpreted by the reader 514.

### Additional Features and Embodiments

Having described various embodiments of articles including cooling features that may be activated to provide cooling and various methods of activating such articles, consider now additional features and embodiments that may be employed with the articles of various embodiments.

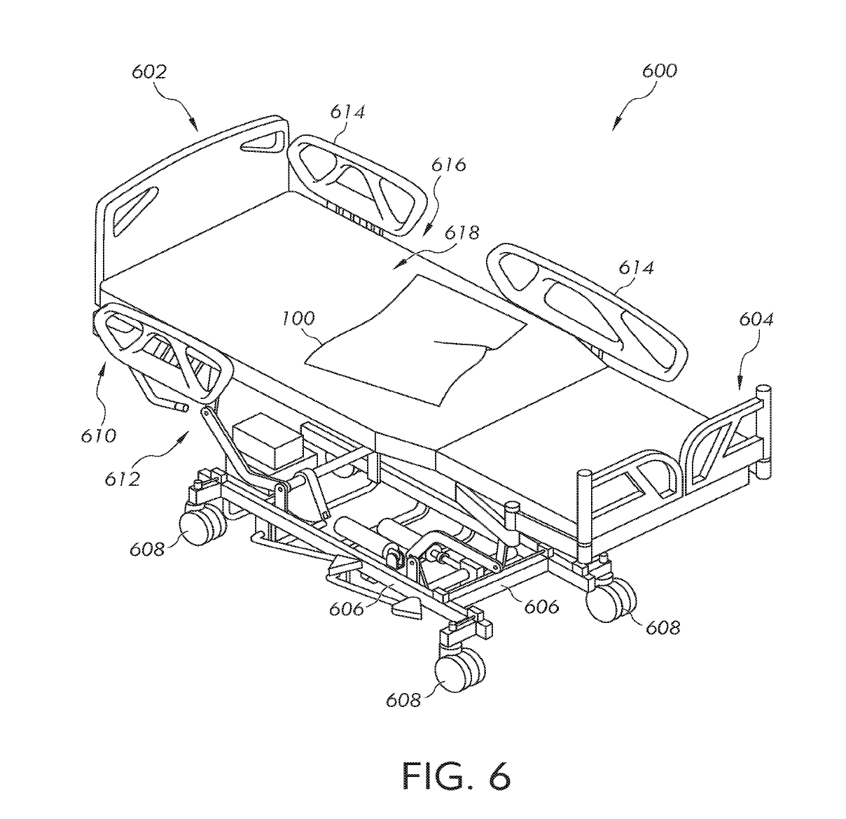

In various embodiments, the cooling article 100 may be positioned on a person support apparatus 600, as depicted in FIG. 6, to provide cooling to an individual. As depicted in FIG. 6, a person support apparatus 600 includes a head end 602 and a foot end 604. The person support apparatus 600 further includes a base frame 606 connected to an intermediate frame 612. Wheels 608 support the base frame 606. A support deck 610 is coupled to the intermediate frame 612. Side rail assemblies 614 are coupled to the support deck 610. A mattress 616 is supported by the support deck 610 and provides a support surface 618 configured to receive an individual (not shown).

As depicted in FIG. 6, the cooling article 100 may be disposed on the support surface 618 such that when an individual is supported by the support surface 618, the cooling article 100 is positioned between the individual and the support surface 618. Although the cooling article 100 is depicted in FIG. 6 as being positioned near the center of the person support apparatus 600, it should be understood that in various embodiments, the cooling article 100 may be positioned toward the head end 602 or toward the foot end 604 of the person support apparatus 600, depending on the particular area of the individual to be cooled.

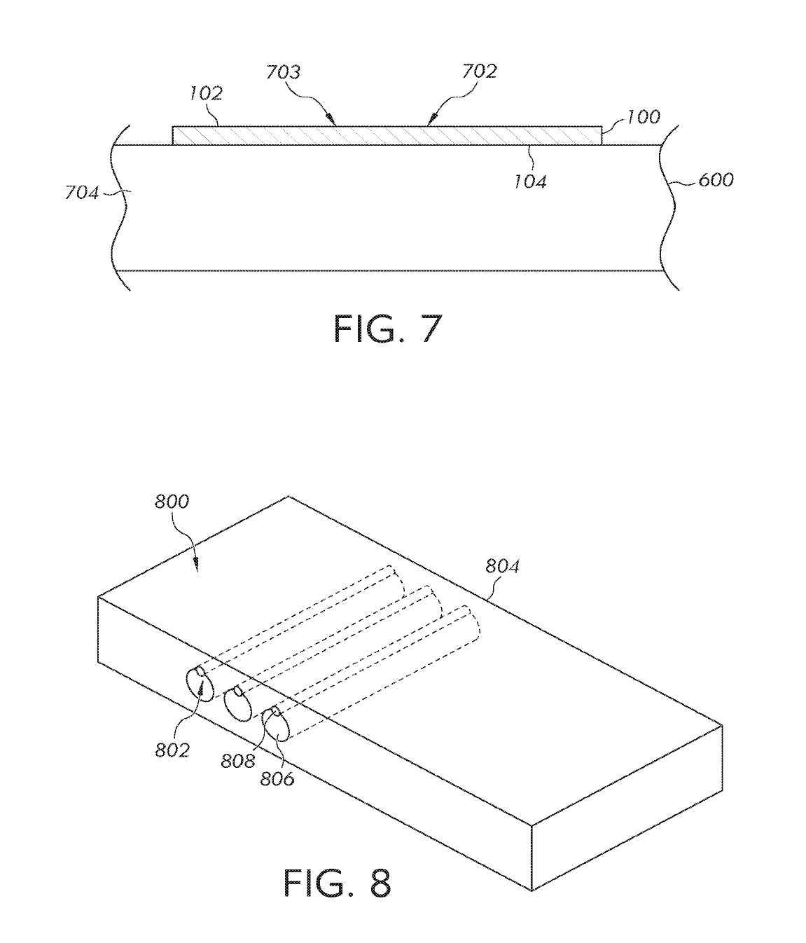

In various embodiments, the person support apparatus 600 may form a heat sink to draw heat away from the cooling article 100. As depicted in FIG. 7, the cooling article 100 is positioned on top of the person support apparatus 600. The first surface 102 of the cooling article 100 forms a support surface 702 while the second surface 104 is in contact with the person support apparatus 600. When coupled to the person support apparatus 600, the cooling article 100 directs heat from a source region 703 proximate the support surface 702 to a sink region 704 formed by the person support apparatus 600.

In embodiments in which the person support apparatus 600 forms a heat sink to draw heat away from the cooling article 100, the cooling article 100 may include one or more flexible thermoelectric devices, such as the embodiment described in accordance with FIG. 3. Accordingly, when an individual is positioned on the support surface 702, heat generated at the source region 703, such as from the individual, may be transferred from the source region 703 to the second surface 104 of the cooling article 100 by the flexible thermoelectric module 323, and the sink region 704 formed by the person support apparatus 600 directs the heat away from the second surface 104.

In embodiments, the person support apparatus 600 may include a low air loss mattress or other mattress that includes one or more air bladders coupled to an air pump or blower. The circulation of air through the mattress enables the person support apparatus 600 to continually remove heat from the second surface 104 of the cooling article 100, and thus, from the source region 703. Various person support apparatuses including convective air mattresses may be employed including, without limitation, those commercially available under the trade names SYNERGY®, ENVISION®, CLINITRON® RITE HITE®, ENVELLA™, and VersaCare A.I.R® from HILL-ROM® or Hill-Rom Services, Inc. (Batesville, Ind.).

In still other embodiments in which the cooling article 100 is coupled to the person support apparatus 600 to enhance cooling, the person support apparatus 600 may include at least one metal surface in contact with the second surface 104 of the cooling article 100. For example, a rail of the person support apparatus 600 or portion of the frame of the person support apparatus 600 may be made of metal that, when in contact with the cooling article 100, draws heat away from the second surface 104 of the cooling article 100 through conduction. In embodiments, instead of metal, the person support apparatus 600 may include another thermally conductive material.

Additional cooling features may be coupled to the person support apparatus to further enhance removal of heat from the source region 703. As a non-limiting example, a cooling source may provide a cooling fluid to the rail of the person support apparatus 600 or the portion of the person support apparatus 600 in contact with the cooling article 100. The cooling source may include, by way of example and not limitation, a pump or fan to direct cooling fluid through the rail of the person support apparatus 600. The cooling fluid absorbs the heat from the rail and the second surface 104 of the cooling article 100 and carries the heat away through the rail, away from the support surface 702, where it may be dissipated in a heat exchanger and/or exhausted to the environment. As used herein, a “cooling fluid” may be any suitable gas or liquid. In embodiments, the cooling fluid may be air, water, or another fluid composition that has high thermal capacity.

As another non-limiting example, thermally conductive fibers may be positioned between the cooling article 100 and the person support apparatus 600. When the cooling article 100 is positioned on the person support apparatus 600, the thermally conductive fibers may be oriented across the person support apparatus 600 in a direction perpendicular to an axis extending from the head end 602 of the person support apparatus 600 to the foot end 604 of the person support apparatus 600 to facilitate coupling the thermally conductive fibers to a cooling fluid and/or a cooling fluid source. In some embodiments, the thermally conductive fibers may extend to an area of the person support apparatus 600 that is not contacted or covered by an individual supported by the support surface 702. The extension of the set of thermally conductive fibers to an area of the person support apparatus 600 that is not in contact with the individual supported by the support surface 702 enables the formation of a temperature gradient along the set of thermally conductive fibers, which in turn enables the set of thermally conductive fibers to function as a cooling mechanism for the cooling article 100. The thermally conductive fibers may include carbon fibers or polymers having a thermal conductivity of greater than about 40 W/m*K. As a non-limiting example, the thermally conductive fibers include pitch-based carbon fiber. Other materials having a suitable thermal conductivity are contemplated.

In various embodiments, the person support apparatus 600 includes a vibration therapy surface 800, as depicted in FIG. 8. The vibration therapy surface 800 may be incorporated into the person support apparatus 600 between the support deck 610 and the mattress 616, or may form the mattress 616, depending on the particular embodiment. The vibration therapy surface 800 illustratively includes three laterally extending percussion and vibration bladders 802 in a back region 804, as shown in FIG. 8. However, more or fewer laterally extending percussion and vibration bladders 802 may be included in various embodiments. Additionally, the percussion and vibration bladders 802 may be located in different regions of the vibration therapy surface 800. For example, one or more of the percussion and vibration bladders 802 may be located near a foot end of the vibration therapy surface 800, near a head end of the vibration therapy surface 800, or near a central support region of the vibration therapy surface 800. It is contemplated that the cooling article 100 (not shown in FIG. 8) may be placed on top of the vibration therapy surface 800, and may be, for example, on top of a mattress 616 disposed on top of the vibration therapy surface 800. Additionally, the cooling article 100 may be positioned near the head end, near the central support region, or near the foot end of the vibration therapy surface, depending on the particular embodiment.

The percussion and vibration bladders 802 depicted in FIG. 8 include a support tube 806 and a percussion tube 808. The dual-tube design of the percussion and vibration bladders 802 provides for supporting an individual while administering percussion and vibration therapy according to known techniques. The support tubes 806 typically maintain a constant pressure while the individual is vibrated as pressurized air is cycled into and out of the percussion tubes 808 at desired frequencies, for example, between 1 Hz and 25 Hz.

In various embodiments, activation of the vibration therapy surface 800 following the exposure of the endothermic ingredient to the endothermic matrix may help to distribute the endothermic ingredient within the endothermic matrix by mixing the reaction components. Accordingly, the vibration therapy surface 800 may be communicatively coupled to a computing device that is configured to activate the vibration therapy surface 800 following activation of the cooling features. For example, a computing device may activate the electrical circuit 408 depicted in FIG. 4B to melt the barrier between the first capsule 402 and the second capsule 404. Following activation of the electrical circuit 408, the computing device may activate the vibration therapy surface 800 to mix the reaction components to generate the endothermic reaction. Vibration therapy surfaces suitable for use in various embodiments, such as the one depicted in FIG. 8, are described in greater detail in U.S. Pat. No. 6,119,291, which is hereby incorporated by reference in its entirety.

Various embodiments described herein include cooling articles that may be used to provide cooling to an individual and methods for activating the same. The cooling articles of various embodiments include cooling features, such as chemical agents that may be mixed to generate an endothermic reaction and/or flexible thermoelectric devices. In addition, various embodiments include one or more features that may be used to enhance the cooling provided by the cooling article, such as by coupling the cooling article to a person support apparatus to further draw heat away from the surface of the cooling article in contact with the individual. The cooling articles described herein may be disposable or may include separable portions that are reusable, such as electronics. Various embodiments further include a color indicator to provide a visual indication that the cooling article has been activated.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

What is claimed is:

1. An article for cooling comprising:
a receptacle;
an endothermic ingredient contained in a first capsule within the receptacle;
an endothermic matrix within the receptacle, wherein the endothermic ingredient, when mixed with the endothermic matrix, generates an endothermic reaction; and
a color indicator, wherein the color indicator is activated upon the generation of the endothermic reaction to indicate that cooling has been initiated.

2. The article for cooling according to claim 1, wherein the color indicator comprises at least one encapsulated dye that is released upon the generation of the endothermic reaction.

3. The article for cooling according to claim 2, wherein the at least one encapsulated dye is encapsulated within the first capsule with the endothermic ingredient.

4. The article for cooling according to claim 2, wherein a capsule containing the at least one encapsulated dye is burst to release the dye.

5. The article for cooling according to claim 4, wherein the capsule is burst responsive to application of pressure or heat.

6. The article for cooling according to claim 2, wherein a capsule containing the at least one encapsulated dye comprises a paraffin or other wax, and wherein the paraffin or other wax is melted to release the at least one encapsulated dye.

7. The article for cooling according to claim 1, wherein the endothermic matrix comprises water and the endothermic ingredient comprises ammonium nitrate.

8. An article for cooling comprising:
a fluid impermeable receptacle;
an endothermic ingredient contained in a first capsule within the fluid impermeable receptacle;
an endothermic matrix within the fluid impermeable receptacle, wherein the endothermic ingredient, when mixed with the endothermic matrix, generates an endothermic reaction; and
an electric circuit configured to melt at least a portion of the first capsule to enable the endothermic ingredient and the endothermic matrix to mix.

9. The article for cooling according to claim 8, wherein the endothermic matrix is contained within a second capsule within the fluid impermeable receptacle, and the electric circuit is further configured to melt at least a portion of the second capsule.

10. The article for cooling according to claim 9, wherein the portion of the first capsule and the portion of the second capsule melted by the electric circuit comprises a barrier positioned between the first capsule and the second capsule.

11. The article for cooling according to claim 8, wherein the electric circuit is removeably coupled to a user interface configured to receive a user input to activate the electric circuit.

12. The article for cooling according to claim 8, wherein the at least the portion of the first capsule is configured to melt at a temperature of from about 54° C. to about 65° C.

13. The article for cooling according to claim 8, wherein the electric circuit comprises an RFID circuit.

14. The article for cooling according to claim 8, wherein the endothermic ingredient and the endothermic matrix are mixed using a vibration system in a person support apparatus upon which the article for cooling is disposed.

15. A system for providing cooling comprising:
a person support apparatus; and
a cooling article comprising a thermoelectric device, wherein the cooling article is coupled to the person support apparatus and directs heat from a source region proximate a support surface of the cooling article to a sink region comprising the person support apparatus.

16. The system of claim 15, further comprising at least one sensor configured to sense a temperature, a moisture level, or a pressure, wherein the at least one sensor provides feedback to the thermoelectric device.

17. The system of claim 16, wherein the at least one sensor is configured to sense a temperature at the support surface and is thermally isolated from a cooling feature of the cooling article.

18. The system of claim 16, wherein the feedback provided to the thermoelectric device by the at least one sensor activates the thermoelectric device.

19. The system of claim 15, wherein the thermoelectric device comprises an integrated coil for receiving radio frequency power.

20. The system of claim 15, wherein the thermoelectric device comprises lead pads configured to connect the thermoelectric device to an external power source.
